(19) United States
(12) Patent Application Publication
(10) Pub. No.: US 2016/0302878 A1
(43) Pub. Date: Oct. 20, 2016

(54) EDGE LIGHTING INSTRUMENTS

(71) Applicant: Novartis AG, Basel (CH)

(72) Inventor: Thomas Kern, Luzern (CH)

(21) Appl. No.: 15/086,540

(22) Filed: Mar. 31, 2016

Related U.S. Application Data
(60) Provisional application No. 62/148,781, filed on Apr. 17, 2015.

(51) Int. Cl. A61B 90/30; A61M 25/00; A61F 9/007; F21V 8/00; A61B 3/00

(52) U.S. Cl. CPC A61B 90/30; G02B 6/001; A61B 3/0008; A61F 9/00736; A61M 25/00; A61B 2090/306

(57) ABSTRACT

Systems, apparatuses, and methods are disclosed that include an optical fiber adapted to emit light along an axial length of an exterior cylindrical surface of the optical fiber. The optical fiber may emit the axially-emitted light over a narrow radial angle δ, such that the axially-emitted light functions in a manner similar to a slit lamp to enhance visibility of nearly transparent structures within the eye. The axially-emitted light may be produced such that no portion of the light is directed in a proximal direction, thereby preventing a portion of the light from being directed at a surgeon.

### TECHNICAL FIELD

The present disclosure relates to apparatuses, systems, and methods associated with illumination devices and, particularly, to ophthalmic illumination devices. The illumination devices are operable to produce a beam of light that essentially functions as a slit lamp to aid in detection of thin, nearly transparent structures within the eye.

### BACKGROUND

The eye is divided into the anterior segment and the posterior segment. The posterior segment includes the vitreous body, which is a clear, colorless, gel-like substance. The vitreous body (also referred to as “vitreous”) occupies approximately two-thirds of the eye's volume.

Various surgical procedures, called vitreoretinal procedures, are commonly performed in the posterior segment of the eye. Vitreoretinal procedures are appropriate to treat many conditions of the posterior segment. Vitreoretinal procedures treat conditions such as age-related macular degeneration (AMD), diabetic retinopathy and diabetic vitreous hemorrhage, macular hole, retinal detachment, epiretinal membrane, CMV retinitis, and many other ophthalmic conditions.

Generally, a surgeon performs vitreoretinal procedures with a microscope and special lenses designed to provide a clear image of the posterior segment. Several tiny incisions just a millimeter or so in length are made on the sclera at the pars plana. The surgeon inserts microsurgical instruments through the incisions such as a fiber optic light source to illuminate inside the eye, an infusion line to maintain the eye's shape during surgery, and instruments to cut and remove the vitreous body.

During such surgical procedures, proper illumination of the inside of the eye is important. An illuminator that may include a thin optical fiber is inserted into the eye to provide the illumination. A light source is used to produce the light carried by the optical fiber into the eye. The light may pass through several optical elements (typically lenses, mirrors, and attenuators) and is passed to the optical fiber. The optical fiber carries the light into the eye.

### SUMMARY

According to one aspect, the present disclosure is directed to an ophthalmic illuminating instrument including a body and an optical fiber extending along the body. The optical fiber may include an exterior cylindrical surface, and the optical fiber may be adapted to emit axially-emitted light from at least a portion of the exterior cylindrical surface along a length of the optical fiber.

According to another aspect, a method of illuminating an ophthalmic surgical area may include inserting an instrument into an eye and illuminating the surgical area with axially-emitted light. The instrument may include a body and an optical fiber extending along the body. The optical fiber may include an exterior cylindrical surface, and the optical fiber adapted to emit axially-emitted light from the exterior cylindrical surface along a length of the optical fiber.

The various aspects of the disclosure may include one or more of the following features. A radial angle of the axially-emitted light may be between 30° and 180°. The radial angle of the axially-emitted light may be between 30° and 40°. The axially-emitted light may be distally-extending axially-emitted light. An axial angle of the distally-extending axially-emitted light may be between 1° and 90°. Essentially all of the light emitted by the optical fiber may be axially-emitted light. The body may include an ophthalmic illuminator. The body may include a vitrectomy cutter. The body may include a cannula. The optical fiber may be disposed in a groove formed in the body. The optical fiber may be adapted to emit a portion of light from a distal end of the optical fiber.

The various aspects of the disclosure may also include one or more of the following features. A radial angle of the axially-emitted light may be between 30° and 180°. The radial angle of the axially-emitted light may be between 30° and 40°.

The OCT probe includes a cannula having a lumen and having a cannula axis. The OCT probe also includes a selectively displaceable light-carrying optical fiber disposed within the lumen and having a distal end. The optical fiber may be adapted to emit light from the distal. A flexor extends through the lumen and includes a first segment and a second segment. The first segment may be coupled to the optical fiber. A driver may be configured to axially displace the second segment such that the optical fiber is laterally displaced. The axially-emitted light may be distally-extending axially-emitted light. An axial angle of the distally-extending axially-emitted light may be between 1° and 90°. Essentially all of the light emitted by the optical fiber may be axially-emitted light. The instrument may include an ophthalmic illuminator. The instrument may include a vitrectomy cutter. The instrument may include a cannula.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 is a perspective view of a portion of an optical fiber emitting axially-emitted light and distally-emitted light.

FIG. 2 shows different axial angles over which the axially-emitted light may be emitted.

FIG. 3 is a perspective view of an instrument that includes an optical fiber providing axially-emitted light and distally-emitted light.

### DETAILED DESCRIPTION

The present disclosure relates generally to illumination devices (interchangeably referred to as “illuminators,” and particularly to ophthalmic illumination devices. The illuminators include an optical fiber that emits light along an entire length or portion thereof separate from any light that may be emitted at a distal end of the optical fiber.

Optical fibers, such as fibers produced by L.E.S.S. SA of Innovation Park, PSE-C CH-1015 Lausanne, Switzerland, are operable to emit light, not only at a distal end of the optical fiber, but also along a length of the fiber. The optical fibers may include nanostructures, such as microscopic breaks or discontinuities within the optical fiber, that causes light traveling along the fiber to exit at one or more locations along their length.

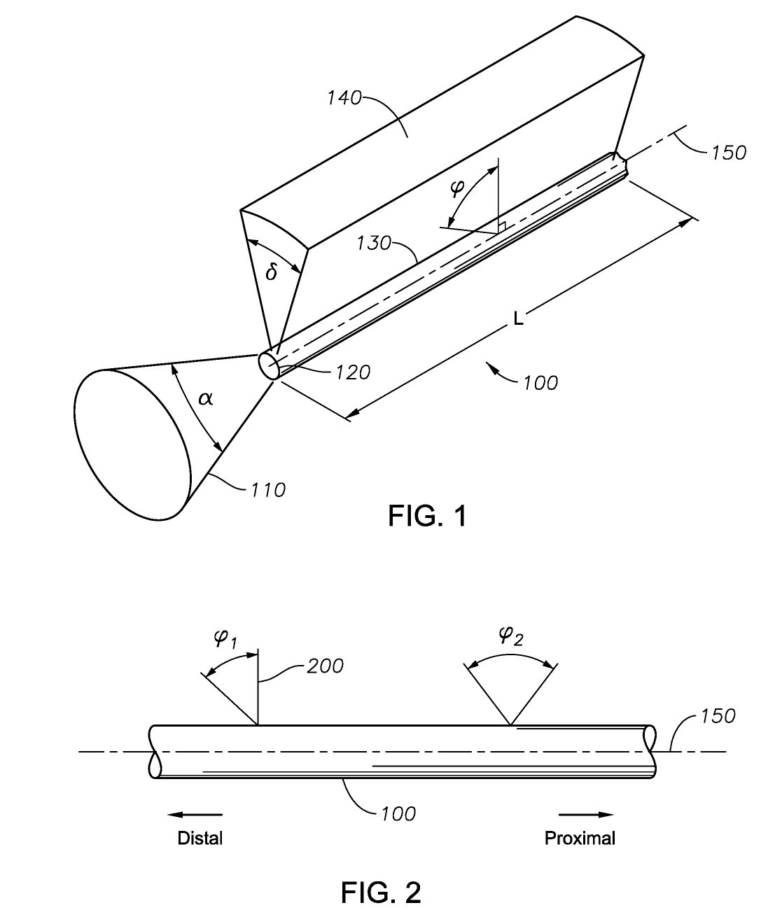

FIG. 1 shows an example optical fiber 100 having an exterior cylindrical surface 130 and a distal end 120. Light 110 is shown being emitted from a distal end 120. The example optical fiber 100 may have a diameter of approximately 200 μm. However, this is provided merely as an example, and the scope of the disclosure is not so limited. Rather, in other implementations, the optical fiber 100 may have a larger or smaller diameter. Light 110 may be emitted in the form of a cone defined by an angle α. Due to a nanostructure of the optical fiber 100, light 140 is emitted along a length L of the optical fiber 100. Light 140 may be defined by a radial angle δ. The discontinuities formed in the optical fiber 100 may be varied so as to vary the angle δ of light 140. Angle δ is measured relative to a plane perpendicular to central axis 150 and about central axis 150.

Generally, light emitted from a distal end of an optical fiber, such as light 110 (referred to as “distally-emitted light”), may be the majority of light emitted from an optical fiber. Further, generally, light emitted from the optical fiber at any other location thereupon is not desirable because such light losses reduce a total amount of light is ultimately emitted from the distal end. However, the light 140 emitted from the exterior cylindrical surface 130 along the length L of optical fiber 100 (referred to as “axially-emitted light”) is emitted at such a narrow angle δ (e.g., 30° to 40°) that the optical fiber 100 functions essentially as a slit lamp. In some instances, the light emitted over an angle δ between 30° to 40° may be limited to a single color bands. However, in other implementations, a broader white light may be possible. For example, a broader white light may be obtained depending on the specification of angle δ and angle φ (discussed in more detail below). An angular range of angle δ and angle φ selected may be dependent upon the wavelength of light being carried by the optical fiber 100.

The narrow beam of light 140 provides illumination to a user, such as a surgeon, that enhances the visibility of nearly transparent structures in the eye, such as vitreous and membranes. Thus, the surgeon is given a better view of the eye's structures so that the surgeon can make better informed decisions, particularly with manipulation of an instrument extending into the eye. The light 140 may be diffracted by the nearly transparent structures, which makes those structures observable to the surgeon. For example, in the course of some vitreoretinal surgeries, removal of the inner limiting membrane (ILM) is necessary. The light 140 emitted form optical fiber 100 enables a surgeon better to detect and remove the ILM without damaging the underlying retinal tissue. Thus, use of the optical fiber 100 significantly reduces risk of injury, including blindness, to a patient.

While the angle δ may be in the range of 30° to 40°, the scope of the disclosure is not so limited. Rather, in other implementations, the angle δ may be larger. For example, the angle δ may be any angle between approximately 30° and 180°. Generally, the narrower the range of angle δ is, the more readily apparent the nearly transparent structures within the eye becomes to a user. The angle a may be in the range of approximately 30 degrees and approximately 150 degrees, between approximately 30 degrees and approximately 120 degrees, between approximately 30 degrees and approximately 90 degrees, and/or other desired or suitable values. Thus, some implementations may be used to provide a larger range of illumination, while other implementations may be used to provide a smaller range of illumination. In some instances, the angle a may be selected to provide targeted illumination for a narrower field of view. In such implementations, the angle a may have a value between approximately 1 degree and approximately 30 degrees, between approximately 10 degrees and approximately 30 degrees, between approximately 20 degrees and approximately 30 degrees, and/or other desired or suitable values.

Another important feature of the optical fiber 100 is axial angle φ of light 1400. Angle φ is defined as an angle in a plane on which the central axis 150 lies or, in cases where the optical fiber 100 is not arranged in a linear fashion, a plane tangent to the central axis 150 of the optical fiber 100. The nanostructures contained within the optical fiber 100 may be configured such that, in the direction axial direction, the light 140 may be emitted perpendicularly and distally. The result is that little, if any, light is emitted proximally and, therefore, towards the surgeon.

Referring to FIG. 2, in the context of φ, axially-emitted light 140 whose angle φ is confined between a line perpendicular to central axis 150, i.e., line 200, and the central axis 150 in a distal direction is considered distally-extending axially emitted light. Similarly, axially-emitted light 140 whose φ extends past the line 200 in a distal direction is considered proximally-extending axially-emitted light.

Again referring to FIG. 2, generally, angle φ may be selected to be essentially any angle between 0° and 180°. For example, in some instances, the angle φ may be any angle between line 200 and the central axis 150 in the distal direction. Thus, the angle φ may be such that none of the light 140 is permitted to extend proximally past line 200. This angle is represented by angle φ₁. Further, neither extent of this angle φ₁ need be coincident with line 200 or the central axis 150. This range of angle corresponds to implementations in which little to no light is emitted proximally. Thus, none of the light 140 is emitted back towards the surgeon. Example angles φ₁ include 1°, 90°, or any angle therebetween.

In other instances, for example, where at least a portion of the light is emitted proximally, the angle φ₂ may be any angle between the extents defined by the central axis 150. This angle is represented by angle φ₂. For example, angle φ₂ may be 1°, 10°, 50°, 95°, 180°, or any other desired angle.

In some instances, an amount of light 110 emitted from the distal end 120 may be substantially reduced or eliminated, thereby resulting all or substantially all of the light traveling through the optical fiber 100 being emitted as light 140. In other instances, a percentage of the light traveling through the optical fiber 100 that is emitted as light 110 and the percentage of light through the optical fiber 100 that is emitted as light 140 may be selected to be a desired amount respectively.

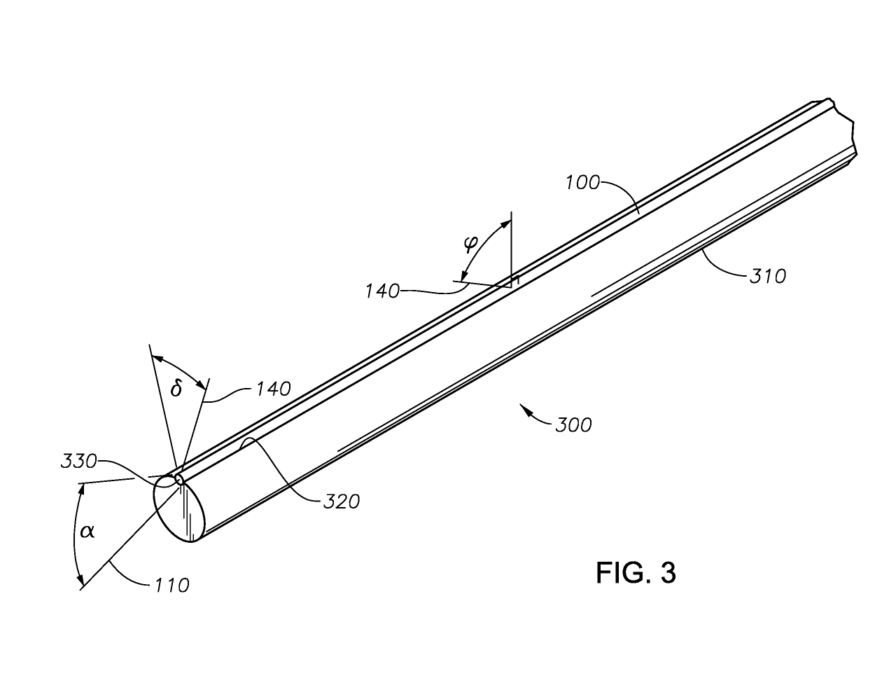

Referring to FIG. 3, optical fiber 100 may form part of an ophthalmic illuminator. The example illuminator 300 comprises a cylindrical body 310 that includes a groove 320 that receives optical fiber 100. In some instances, the illuminator 300 may be a dedicated illuminator device. In the example shown, the illuminator emits both light 110 from distal end 330 and light 140 emitted along a length of the optical fiber 100. As explained above, an angle a may be selected over which light 110 is emitted, and light 140 may have radial and axial angles δ and φ, respectively, as desired. Although a single optical fiber is indicated, the illuminator 300 may include a plurality of optical fibers 100.

While FIG. 3 provides an example in which an optical fiber as described herein is disposed in a dedicated illumination device, the scope of the disclosure is not so limited. Rather, one or more optical fibers as described herein may be incorporated into other instruments. For example, an optical fiber, such as optical fiber 100, may be incorporated into a cannula, a vitrectomy cutter, an infusion line, grasping instrument (e.g., forceps), cutting instruments (e.g., scissors), or any other desired instrument. The axially-emitted light provided by the optical fiber provides improved visualization of a surgical area, thereby improving patient safety and potentially reducing an amount of time required to complete a surgical procedure.

Although the disclosure provides numerous examples, the scope of the present disclosure is not so limited. Rather, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An ophthalmic illuminating instrument comprising:
a body;
an optical fiber extending along the body, the optical fiber comprising an exterior cylindrical surface, and the optical fiber adapted to emit axially-emitted light from at least a portion of the exterior cylindrical surface along a length of the optical fiber.

2. The ophthalmic illuminating instrument of claim 1, wherein a radial angle of the axially-emitted light is between 30° and 180°.

3. The ophthalmic illuminating instrument of claim 2, wherein the radial angle of the axially-emitted light is between 30° and 40°.

4. The ophthalmic illuminating instrument of claim 1, wherein the axially-emitted light is distally-extending axially-emitted light.

5. The ophthalmic illuminating instrument of claim 4, wherein an axial angle of the distally-extending axially-emitted light is between 1° and 90°.

6. The ophthalmic illuminating instrument of claim 1, wherein essentially all of the light emitted by the optical fiber is axially-emitted light.

7. The ophthalmic illuminating instrument of claim 1, wherein the body comprises an ophthalmic illuminator.

8. The ophthalmic illuminating instrument of claim 1, wherein the body comprises a vitrectomy cutter.

9. The ophthalmic illuminating instrument of claim 1, wherein the body comprises a cannula.

10. The ophthalmic illuminating instrument of claim 1, wherein the optical fiber is disposed in a groove formed in the body.

11. The ophthalmic illuminating instrument of claim 1, wherein the optical fiber is adapted to emit a portion of light from a distal end of the optical fiber.

12. A method of illuminating an ophthalmic surgical area comprising:
inserting an instrument into an eye, the instrument comprising:
a body; an
an optical fiber extending along the body, the optical fiber comprising an exterior cylindrical surface, and the optical fiber adapted to emit axially-emitted light from the exterior cylindrical surface along a length of the optical fiber; and
illuminating the surgical area with the axially-emitted light.

13. The method of claim 12, wherein a radial angle of the axially-emitted light is between 30° and 180°.

14. The method of claim 13, wherein the radial angle of the axially-emitted light is between 30° and 40°.

15. The method of claim 12, wherein the axially-emitted light is distally-extending axially-emitted light.

16. The method of claim 15, wherein an axial angle of the distally-extending axially-emitted light is between 1° and 90°.

17. The method of claim 1, wherein essentially all of the light emitted by the optical fiber is axially-emitted light.

18. The method of claim 1, wherein the instrument comprises an ophthalmic illuminator.

19. The method of claim 1, wherein the instrument comprises a vitrectomy cutter.

20. The method of claim 1, wherein the instrument comprises a cannula.
